(19) United States
(12) Patent Application Publication
(10) Pub. No.: US 2009/0099651 A1
(43) Pub. Date: Apr. 16, 2009

(54) LIPID COATINGS FOR IMPLANTABLE MEDICAL DEVICES

(75) Inventors: Dorna Hakimi-Mehr, Vancouver (CA); Mark Landy, Atlanta, GA (US); Vlad Budzynski, North Vancouver (CA); Michael N.C. Chen, Coquitlam (CA); Aleksy Tsetkov, Richmond (CA); Manus Tsui, Richmond (CA); Quanzu Yang, Vancouver (CA)

Correspondence Address: RISSMAN JOBSE HENDRICKS & OLIVERIO, LLP, 100 Cambridge Street, Suite 2101, BOSTON, MA, 02114, US

(73) Assignee: MIV Therapeutics, Inc., Vancouver (CA)

(21) Appl. No.: 12/060,604

(22) Filed: Apr. 1, 2008

Related U.S. Application Data
(60) Provisional application No. 60/978,988, filed on Oct. 10, 2007.
(60) Provisional application No. 60/981,273, filed on Oct. 19, 2007.

(51) Int. Cl. A61L 27/54; A61F 2/82

(52) U.S. Cl. 623/1.42; 623/1.46

(57) ABSTRACT

Disclosed herein are medical devices, such as stents, comprising a porous substrate, and a composition coating and/or impregnating the porous substrate where the composition comprises a bioresorbable carrier (e.g., at least one lipid) and at least one pharmaceutically active agent.

### RELATED APPLICATION

This application claims the benefit of priority under 35 U.S.C. §119(e) of U.S. Provisional Application No. 60/978,988, filed Oct. 10, 2007, and U.S. Provisional Application No. 60/981,273, filed Oct. 19, 2007, the disclosures of which are incorporated herein by reference.

### FIELD OF THE INVENTION

Disclosed herein are coatings for medical devices, such as implantable medical devices (e.g., stents), and processes for making the same. The stent comprises a porous substrate having pores coated or impregnated with a composition comprising one or more lipids and one or more therapeutic agents.

### BACKGROUND OF THE INVENTION

Implantable medical devices are used in a wide range of applications including bone and dental replacements and materials, vascular grafts, shunts and stents, and implants designed solely for prolonged release of drugs. The devices may be made of metals, alloys, polymers or ceramics.

Arterial stents have been used for many years to prevent restenosis after balloon angioplasty (expanding) of arteries narrowed by atherosclerosis or other conditions. Restenosis involves inflammation and the migration and proliferation of smooth muscle cells of the arterial media (the middle layer of the vessel wall) into the intima (the inner layer of the vessel wall) and lumen of the newly expanded vessel. This migration and proliferation, as well production of extracellular matrix by smooth muscle cells, is called neointima formation. The inflammation is at least partly related to the presence of macrophages. The macrophages are also known to secrete cytokines and other agents that stimulate the abnormal migration and proliferation of smooth muscle cells. Stents reduce but do not eliminate restenosis.

Drug eluting stents have been developed to elute anti-proliferative drugs from a non-degradable polymer coating and are currently used to further reduce the incidence of restenosis. Examples of such stents are the Cypher® stent, which elutes sirolimus, and the Taxus® stent, which elutes paclitaxel. Recently it has been found that both of these stents, though effective at preventing restenosis, cause potentially fatal thromboses (clots) months or years after implantation. Late stent thrombosis is thought to be due to the persistence of the somewhat toxic drug or the polymer coating or both on the stent for long time periods. Examination of some of these stents removed from patients frequently shows no covering of the stent by the vascular endothelial cells of the vessel intima. This is consistent with the possible toxicity of the retained drugs or non-degradable polymer. The lack of endothelialization may contribute to clot formation.

There have been attempts to develop polymer-free coatings. However, these approaches have failed to produce the desired outcomes due to problems such as lack of mechanical integrity necessary to undergo device preparation and implantation, and may also result in undesirably fast release of the therapeutic agent.

Accordingly, there remains a need to develop new drug eluting stents having sufficient efficacy, mechanical integrity, and a surface that is biocompatible.

### SUMMARY OF THE INVENTION

One embodiment provides a stent comprising:

a porous substrate; and

at least one composition impregnating at least a portion of the porous substrate, wherein the composition comprises at least one pharmaceutically effective agent and at least one lipid.

Another embodiment provides a medical device, comprising at least one coating covering at least a portion of the device, the at least one coating comprising:

a porous substrate;

a composition impregnating the porous substrate, the composition comprising at least one pharmaceutically effective agent and at least one lipid selected from fatty acids, fatty amines, and neutral lipids.

Another embodiment provides a stent comprising at least one coating covering at least a portion of the device, the at least one coating comprising:

a porous substrate;

a composition coating and/or impregnating the porous substrate, the composition comprising at least one pharmaceutically effective agent and at least one lipid.

Another embodiment provides a method of treating at least one disease or condition comprising:

implanting in a subject in need thereof a stent comprising at least one coating covering at least a portion of the device, the at least one coating comprising:
a porous substrate;
a composition coating or impregnating the porous substrate, the composition comprising at least one pharmaceutically effective agent and at least one lipid; and

releasing from the device the at least one pharmaceutically active agent.

In one embodiment, the at least one pharmaceutically active agent is released from the device associated with particles comprising the at least one lipid, wherein the particles are selected from liposomes, nanocapsules, microcapsules, microdroplets, nanodroplets, microspheres, nanospheres, and micelles. In one embodiment, the composition further comprises at least one surfactant, including any surfactant disclosed herein.

Another embodiment provides a method of treating at least one disease or condition comprising:

implanting in a subject in need thereof a medical device comprising at least one coating covering at least a portion of the device, the at least one coating comprising:
a porous substrate;
a composition impregnating the porous substrate, the composition comprising at least one pharmaceutically effective agent and at least one lipid selected from fatty acids, fatty amines, and neutral lipids; and
releasing from the device the at least one pharmaceutically active agent.

### BRIEF DESCRIPTION OF THE DRAWINGS

FIG. 1 is a schematic of a device coated with a porous substrate impregnated with a composition comprising at least one lipid and at least one pharmaceutically active agent;

FIGS. 2A, 2B, and 2C are photographs of a coated stent as described in Example 2;

FIG. 3 is a release curve plotting cumulative % drug release (y-axis) versus time of elution (days, x-axis) for a coated prior art device as described in Example 3;

FIG. 4 is a release curve cumulative % drug release (y-axis) versus time of elution (days, x-axis) for a stent as described in Example 3;

FIG. 5A is a photograph of porcine lower anterior descending (LAD) coronary artery section indicating the typical histology of the implanted Cypher™ stent, as described in Examples 4 and 5; and

FIG. 5B is a photograph of a porcine LAD showing a coronary artery section and the histology of an implanted stent, as described in Examples 4 and 5.

### DETAILED DESCRIPTION

Disclosed herein are coatings for medical devices, such as implantable medical devices, e.g., stents. One embodiment provides a medical device, such as a stent, comprising:

a porous substrate; and

a composition impregnating at least a portion of the porous substrate, wherein the composition comprises at least one pharmaceutically effective agent and a bioresorbable carrier.

In one embodiment, the porous substrate can have pores and voids sufficiently large enough to contain a drug yet have passageways that, when exposed to an aqueous solution, permit the drug to be released from the pores of the substrate and enter the aqueous solution. In one embodiment, “aqueous solution” refers to an in vitro solution comprising water and optionally including buffers and/or other components, such as those components that adjust the solution to a desired pH. In another embodiment, the aqueous solution is a body fluid.

The size and volume fraction of the substrate porosity can also be adjusted to influence the release rate of the therapeutic agent, e.g., by adjusting the porosity volume and/or pore diameter. For example a porous substrate possessing nano-size porosity is expected to decrease the release rate of the therapeutic agent compared to a porous substrate having micro-size porosity. A porous substrate, e.g., a porous ceramic, may also aid in providing the coating with sufficient flexibility where the device is a stent.

In one embodiment, the porous substrate is the medical device or the stent itself. The stent can be made of various materials including stainless steel, CoCr, titanium, titanium alloys, NiTi. The stent can be made of a polymer, e.g., polymers having 10 or more covalently bonded monomers or comonomers. In one embodiment, the polymer is selected from those typically used for implantable medical devices. Exemplary polymers include polyurethanes, polyacrylate esters, polyacrylic acid, polyvinyl acetate, silicones, styrene-isobutylene-styrene block copolymers such as styrene-isobutylene-styrene tert-block copolymers (SIBS); polyvinylpyrrolidone including cross-linked polyvinylpyrrolidone; polyvinyl alcohols, copolymers of vinyl monomers such as EVA; polyvinyl ethers; polyvinyl aromatics; polyethylene oxides; polyesters including polyethylene terephthalate; polyamides; polyacrylamides; polyethers including polyether sulfone; polyalkylenes including polypropylene, polyethylene and high molecular weight polyethylene; polycarbonates, siloxane polymers; cellulosic polymers such as cellulose acetate; polymer dispersions such as polyurethane dispersions (BAYHDROL®); squalene emulsions; poly(n-butyl methacrylate)/poly(ethene vinyl acetate), polyacrylate, poly(lactide-co-E-caprolactone), phosphorylcholine, PTFE, paralyene C, polyethylene-co-vinyl acetate, poly n-butylmethacrylate, poly(styrene-b-isobutylene-b-styrene) (a tri-block copolymer of styrene and isobutylene subunits built on 1,3-di(2-methoxy-2-propyl)-5-tert-butylbenzene, Transelute™), and mixtures and copolymers of any of the foregoing.

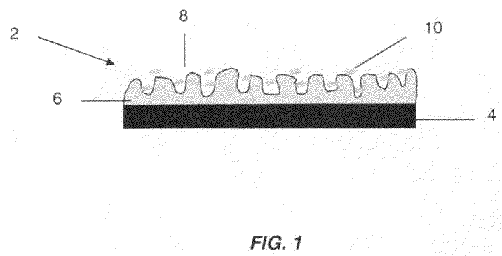

In another embodiment, the porous substrate comprises a material that covers at least a portion of the stent. FIG. 1 schematically depicts one embodiment of the coated devices disclosed herein. “Coated medical device” as used herein includes those devices having one or more coatings, i.e., at least one coating. The at least one coating can comprise one coating covering at least a portion of the device, e.g., all or some of the device. For example, where the device is a stent, the coating can cover the entire stent, or can cover only the portion of the stent that contacts a body lumen, or any other selected portion. The device may employ more than one coating for different portions of the device, or can employ multiple layers of coatings.

In FIG. 1, a section of device 2 comprises surface 4 coated with a porous substrate 6, the surface of which is schematically depicted. Impregnating substrate 6 is a composition comprising a pharmaceutically active agent 10 in a bioresorbable carrier 8 that acts as a vehicle for the active agent. The carrier 8 can be one or more lipids, or any other bioresorbable carrier disclosed herein. The agent 10 may contact the porous substrate 6, or may be suspended in the carrier 8 (e.g., lipid(s)) without contacting substrate 6. The agent 10 may be embedded in the carrier 8 in molecular or particulate form.

In one embodiment, the device can be prepared by initially coating the device with substrate 6, followed by coating the device with the composition comprising carrier (e.g., lipid(s)) 8 and agent (10). In another embodiment, a therapeutic agent can be co-deposited with a porous substrate coating using an electrodeposition method (e.g., in the codeposition of ceramics such as calcium phosphates). For example, the therapeutic agent(s) dissolved in the electrolyte solution can be co-deposited with the substrate coating. Multiple layers can be envisioned by repeating any of the disclosed layering processes as desired to form a porous biocompatible coating, containing multiple layers of formulations containing multiple therapeutic agents. Each layer may contain one or more agents, which can be the same or different depending on the desired drug course.

As disclosed herein, instead of a porous substrate 6 that coats the stent, the stent itself can comprise a porous substrate in which the carrier and active agent impregnates at least a portion thereof.

In one embodiment, the bioresorbable carrier comprises at least one lipid. Accordingly, another embodiment provides a stent, comprising:

a porous substrate;

a composition impregnating at least a portion of the porous substrate, wherein the composition comprises at least one pharmaceutically effective agent and at least one lipid.

The pharmaceutically acceptable agent can be combined with the at least one lipid using any method known in the art. In one embodiment, the at least one lipid is dissolved in a first solvent and the agent is dissolved in a second solvent where the first and second solvents are the either miscible or the same (in this case, the lipid(s) and agent can alternatively be dissolved in a solvent to form a single solution). The lipid-containing solution can then combined with drug-containing solution to achieve a pre-determined percentage of the therapeutic agent and lipid. In one embodiment, the percentage of the agent in the composition can vary from 1% to 90%, e.g., from 1% to 50%, from 1% to 25%, from 1% to 10%, or from 1% to 5%.

The viscosity may be controlled as desired to facilitate impregnation of the composition into the porous substrate and/or contain the composition on the surface of the stent until after implantation. In one embodiment, the viscosity of the lipid/drug-containing solution can be adjusted by adjusting the concentrations of the first and second solutions. For example, low concentrations of lipid-containing solution and drug-containing solution can yield a low concentration of the lipid/drug solution, which in turn can possess low viscosity (relative to a higher concentration solution). In one embodiment, the lipid-containing solution has a concentration of at least 5% (w/w), or at least 10% (w/w), and the drug-containing solutions has a concentration of at least 2% (w/w), or at least 4% (w/w). In one embodiment, the lipid-containing solution has a concentration of 10% (w/w) and the drug-containing solution has a concentration of 4% (w/w).

In one embodiment, the at least one pharmaceutically active agent is dissolved in a solvent, and the at least one lipid combined with this solution to achieve a pre-determined percentage of the agent in the lipid. The concentration of drug-containing solution may determine the viscosity of the final drug/lipid solution. Alternatively, the at least one lipid is dissolved in a solvent, and the at least one pharmaceutically active agent is combined with this solution to achieve a pre-determined percentage of the agent in the lipid. The concentration of solution lipid-containing solution may determine the viscosity of the final drug/lipid solution.

In one embodiment, the at least one pharmaceutically active agent can be combined with the at least one lipid in particulate form. For example, the therapeutic agent in powder form can be directly combined with the at least one lipid. The mixture can be further homogenized by using a homogenizer or with an ultrasound device to achieve a uniform mixture. The homogenized mixture can be applied to the porous substrates using known techniques in the art, such as any one or more of the techniques disclosed herein.

In embodiments where at least one of the pharmaceutically active agents and the at least one lipid are not miscible (e.g. the agent is hydrophilic), the lipid(s) and agent(s) can be mixed by using a w/o (water-in-oil) emulsion technique. For example, the agent(s) can be dissolved in water or another hydrophilic solvent. The lipid(s) can be dissolved in a second solvent. If the drug-containing and lipid-containing solutions are miscible, they can be simply mixed to form a drug/lipid-containing solution that achieve a pre-determined percentage of the agent in the lipid. If the solutions are not miscible, the drug-containing solution can be combined with the lipid-containing solution to form an emulsion. The emulsion can be subjected to ultra-sonication to homogenize the emulsion. In one embodiment, one or more surfactants can be combined with the emulsion to stabilize the emulsion. The surfactant(s) can be ionic or nonionic. Exemplary ionic surfactants include chitosan, didodecyldimethylammonium bromide, and dextran salts, e.g., naturally occurring ionizable dextrans such as dextran sulfate or dextrans synthetically modified to contain ionizable functional groups. Exemplary nonionic surfactants include dextrans, polyoxyethylene castor oil, polyoxyethylene 35 soybean glycerides, glyceryl monooleate, triglyceryl monoleate, glyceryl monocaprylate, glycerol monocaprylocaprate, propylene glycol monolaurate, triglycerol monooleate, stearic glycerides, sorbitan monostearate (Span® 60), sorbitan monooleate (Span® 80), polyoxyethylene sorbitan monolaurate (Tween® 20), polyoxyethylenesorbitan tristearate (Tween® 65), and polyoxyethylene sorbitan monooleate (Tween® 80).

The lipid/drug solution can be applied to the porous substrate by using techniques known in the art, such as spraying, dipping, rolling, or brushing. In one embodiment, the lipid/drug solution is applied by dipping under vacuum a device coated with the porous substrate. In another embodiment, after dipping, the device is further subjected to a spinning process to remove the excess lipid/drug solution on the surface of the coated device.

After the completion of the coating process, residual solvents can be removed using techniques known to the art, such as by applying heat, vacuum, or drying at room temperature, e.g., in air. In one embodiment the coated device is placed under vacuum to remove residual solvents. In one embodiment, the coated medical device can be placed under vacuum conditions or any other atmosphere where the device has minimal exposure to humidity (e.g., in a desiccator).

In one embodiment, the coated device is allowed to stand for a period of time to stabilize the coating, which may improve the reproducibility of the drug release profile. For example, certain non-stabilized coatings may produce burst-like elution curves (e.g., more than 30% of the initial drug content of the coating is released within 24 hours). In one embodiment, the coating is stabilized for at least 1 week, at least two weeks, at least three weeks, or at least one month. In one embodiment, the coated device is stabilized under conditions in which the coating is exposed to minimal humidity. Coatings that have been stabilized can result in reproducible elution curves and reduce the burst-like behavior.

In one embodiment, the coating is capable of sustained drug delivery. In one embodiment, at least 50% of the pharmaceutically active agent is released from the porous substrate over a period ranging from 7 days to 6 months, from 7 days to 3 months, from 7 days to 2 months, from 7 days to 1 month, from 10 days to 1 year, from 10 days to 6 months, from 10 days to 2 months, from 10 days to 1 month, or from 30 to 40 days.

In one embodiment, the porous substrate is selected from ceramics, such as those ceramics known in the art to be biocompatible, e.g., metal oxides such as titanium oxide, aluminum oxide, silica, and indium oxide, metal carbides such as silicon carbide, and one or more calcium phosphates such as hydroxyapatite, octacalcium phosphate, α- and β-tricalcium phosphates, amorphous calcium phosphate, dicalcium phosphate, calcium deficient hydroxyapatite, and tetracalcium phosphate.

One embodiment provides a metal stent comprising at least one coating covering at least a portion of the stent, where the at least one coating comprises a porous calcium phosphate. Calcium phosphates may be used to coat devices made of metals or polymers to provide a more biocompatible surface. Calcium phosphates are often desirable because they occur naturally in the body, are non-toxic and non-inflammatory, and are bioabsorbable. Such devices or coatings may serve as a matrix for cellular and bone in-growth in orthopedic devices or to control the release of a therapeutic agent from any device. In the field of vascular stents, calcium phosphate coatings can be attractive because they can provide a biocompatible surface that can be rapidly covered by the endothelial cells of the vascular intima.

In one embodiment, the coating is a hydroxyapatite coating. Hydroxyapatite typically constitutes 70% of natural bone composition and can afford good biocompatibility. It has been demonstrated that hydroxyapatite invokes minimal or no inflammatory reaction or foreign body response. A porous hydroxyapatite layer can be deposited on the surface of the medical device using a variety of techniques as disclosed herein.

In one embodiment, the carrier, e.g., the at least one lipid, is in pliable form that serves as a water-insoluble vehicle for the at least one pharmaceutically active agent. The carrier (e.g., lipid(s)) can help contain the agent in the pores of the substrate and/or it can aid its release from the substrate. In one embodiment, the carrier (e.g., lipid(s)) is a biodegradable and can release an agent by slow dissolution, biodegradation, or slow release of the agent. In another embodiment, the lipid can also help control the release of drug by retarding or increasing the rate of release depending on the relative miscibility of the lipid and drug. In another embodiment, the drug can be released from the porous substrate in which the lipid takes the form of particles such as capsules (nanocapsules, microcapsules), droplets (microdroplets, nanodroplets), spheres (microspheres, nanospheres), and/or micelles. In one embodiment, the release of particles is aided by the addition of at least one surfactant to the composition. The at least one surfactant can be any of the ionic or nonionic surfactants disclosed herein. In one embodiment, the drug is encapsulated in the lipid particles. In another embodiment, the drug is released from the coating while dissolved, dispersed, or otherwise attached to the lipid particles. Such drug/lipid particles may enhance the uptake of the therapeutic agent by the cells and/or increase the residence time of the drug in the surrounding tissue by reducing the solubility of the therapeutic agent in the physiological fluids, either of which may improve the potency of the drug.

In one embodiment, the device is a stent, and the composition comprising the lipid(s) and pharmaceutically active agent(s) can be deposited in a variety of forms that either impregnate or coat the porous substrate. Accordingly, one embodiment provides a stent comprising at least one coating covering at least a portion of the device, the at least one coating comprising:

a porous substrate;

a composition coating and/or impregnating the porous substrate, the composition comprising at least one pharmaceutically effective agent and at least one lipid.

In one embodiment, the composition is in the form of films, liposomes nanocapsules, microcapsules, microdroplets, nanodroplets, microspheres, nanospheres, micelles, and combinations thereof. In another embodiment, the composition is released from the stent in the form of films, liposomes nanocapsules, microcapsules, microdroplets, nanodroplets, microspheres, nanospheres, micelles, and combinations thereof.

In one embodiment, the stent, when implanted, releases the pharmaceutically active agent(s) associated with lipid-based particles. In one embodiment, the pharmaceutically active agent(s) are encapsulated in the particles. The particles can take the form of liposomes, nanocapsules, microcapsules, microdroplets, nanodroplets, microspheres, nanospheres, micelles, and combinations thereof.

In some instances, macrophages can take up certain particles having a diameter of about 1-2 μm or greater. Lipid-based particles can be designed to have a diameter ranging from of about 1-2 μm and greater in order to increase their uptake by macrophages and reduce inflammation, such as the inflammation component of restenosis. In one embodiment the composition releases therapeutic agent-containing particles (e.g., capsules (nanocapsules, microcapsules), droplets (microdroplets, nanodroplets), spheres (microspheres, nanospheres), and/or micelles) having a diameter of about 1-2 μm or greater to inhibit macrophages and prevent inflammation. In one embodiment, at least 5%, at least 10% or at least 25% of the particles have a diameter of about 1-2 μm or greater, thereby increasing the likelihood of uptake by macrophages.

The particle size distribution can allow the drug to be released in different forms and can enable the drug to exhibit dual functionality: (1) the drug associated with particles having a diameter of greater than 1 or 2 μm can be taken up by macrophages to treat a first condition, such as an inflammatory reaction, and (2) the same drug in free form or associated with particles less than 1 or 2 μm can treat a second condition, e.g., proliferation. In one embodiment, for the treatment of restenosis, a drug known for being an antiproliferative agent can be released associated with a particle greater than 1 or 2 μm to reduce the number of inflammatory agents produced by macrophages whereas the free form of the drug or the drug associated with particles less than 1 or 2 μm can act to inhibit proliferation of smooth muscle cells.

The lipid/drug composition can be deposited in or on the substrate in number of ways. In one embodiment, the at least one lipid is dissolved in a first solvent and the agent is dissolved in a second solvent where the first and second solvents are either miscible or the same (in this case, the lipid(s) and agent can alternatively be dissolved in a solvent to form a single solution). The lipid-containing solution can be then combined with drug-containing solution to achieve a solution with a pre-determined percentage of the therapeutic agent and lipid. This solution can be formed into micro/nano spheres using methods known in the art and can be deposited in or on the porous substrate. In one example, the solution can be added to an aqueous solution (e.g., an o/w oil-in-water emulsion) and can be homogenized to produce micro/nanospheres of lipid containing the drug. The homogenized composition can be then deposited into the porous substrate through spraying, dipping, dip and spin or any other method known in the art. In another embodiment the emulsion can be filtered to produce micro/nanospheres of desired size. The micro/nanospheres can then be suspended in another solvent or solution and be deposited into substrate using methods known in the art such as spraying, dip, or dip and spin. Upon exposure to an aqueous solution (e.g., body fluids) the micro/nanospheres can be resuspended in the liquid surrounding the stent, encapsulating the drug, and be taken up by macrophages or other types of cells.

The agent in the porous substrate can be hydrophilic, hydrophobic, or amphipathic. In one embodiment the agent impregnating the porous substrate is soluble in the at least one lipid. In another embodiment the agent is insoluble in the at least one lipid.

The at least one lipid can be neutral or charged. Neutral lipids include monoglycerides, diglycerides, triglycerides, ceramides, sterols, sterol esters, waxes, tocopherols, monoalkyl-diacylglycerols, fatty alcohols comprising a hydrocarbon chain of at least 8 carbon atoms (e.g., C₈-C₃₀ fatty alcohols, or a hydrocarbon chain of at least 12 carbon atoms, e.g., C₁₂-C₃₀ fatty alcohols), N-monoacylsphingosines, N,O-diacylsphingosines, and triacylsphingosines. In one embodiment, the monoglycerides, diglycerides, and triglycerides are derived from fatty acids having a chain length of at least 4 carbon atoms, such as a chain length of at least 8 carbon atoms, or a chain length of at least 12 carbon atoms.

In one embodiment, the at least one lipid is selected from vegetable oils, animal oils, and synthetic lipids. In one embodiment, the at least one lipid is selected from triglycerides and vegetable oils.

Charged lipids include phospholipids, fatty acids and fatty amines. Exemplary phospholipids include diacylglycerophosphates, monoacylglycerophosphates, cardiolipins, plasmalogens, sphingolipids and glycolipids. Fatty acids and fatty amines may have a chain length of at least 8 carbon atoms, or a chain length of at least 12 carbon atoms.

Lipids are insoluble or sparingly soluble in water. In one embodiment, no more than 10% by weight of the at least one lipid is soluble in water, e.g., no more than 5% by weight of the at least one lipid is soluble in water, no more than 3% by weight of the at least one lipid is soluble in water, no more than 1% by weight of the at least one lipid is soluble in water, or no more than 0.1% by weight of the at least one lipid is soluble in water

Exemplary lipids include soybean oil, cottonseed oil, rapeseed oil, sesame oil, corn oil, peanut oil, safflower oil, fish oil, triolein, trilinolein, tripalmitin, tristearin, trimyristin, triarachidonin, azone, castor oil, cholesterol, and cholesterol derivatives such as cholesteryl oleate, cholesteryl linoleate, cholesteryl myristate, cholesteryl palmitate, cholesteryl arachidate.

In one embodiment, the at least one lipid is selected from fatty acids, fatty amines, and neutral lipids.

In one embodiment, in addition to the at least one lipid, the composition further comprises at least one additional lipid. Exemplary additional lipids include phospholipids, glycolipids, sphingomyelins, cerebrosides, gangliosides, and sulfatides.

Examples of these types of lipids and other lipids are disclosed in U.S. Provisional Application No. 60/952,565, filed Jun. 7, 2007, the disclosure of which is incorporated herein by reference.

The at least one pharmaceutically active agent may be anti-inflammatory agents, anti-proliferatives, pro-healing agents, gene therapy agents, extracellular matrix modulators, anti-thrombotic agents, anti-platelet agents, anti-neoplastic agents, anti-angiogenic agents, antiangioplastic agents, antisense agents, anticoagulants, antibiotics, bone morphogenetic proteins, integrins (peptides), and disintegrins (peptides and proteins) inhibitors of restenosis, smooth muscle cell inhibitors, immunosuppressive agents, anti-angiogenic agents, paclitaxel, sirolimus, everolimus, tacrolimus, biolimus, pimecrolimus, midostaurin, bisphosphonates (e.g., zoledronic acid), heparin, gentamycin, or imatinib mesylate (gleevec).

Exemplary anti-inflammatory agents include pimecrolimus, adrenocortical steroids (e.g., cortisol, cortisone, fludrocortisone, prednisone, prednisolone, 6α-methylprednisolone, triamcinolone, betamethasone, and dexamethasone), non-steroidal agents (salicylic acid derivatives such as aspirin, para-aminophenol derivatives such as acetaminophen, indole and indene acetic acids (e.g., indomethacin, sulindac, and etodalac), heteroaryl acetic acids (e.g., tolmetin, diclofenac, and ketorolac), arylpropionic acids (ibuprofen and derivatives), anthranilic acids (mefenamic acid, and meclofenamic acid), enolic acids (piroxicam, tenoxicam, phenylbutazone, and oxyphenthatrazone). Exemplary anti-proliferatives include sirolimus, everolimus, actinomycin D (ActD), taxol, paclitaxel, and midostaurin. Exemplary pro-healing agents include estradiol. Exemplary gene therapy agents include gene delivering vectors e.g., VEGF gene, and c-myc antisense. Exemplary extracellular matrix modulators include batimastat. Exemplary anti-thrombotic agents/anti-platelet agents include sodium heparin, low molecular weight heparin, hirudin, argatroban, forskolin, vapiprost, prostacyclin and prostacyclin analogs, dextran, D-phe-pro-arg-chloromethylketone (e.g., synthetic antithrombin), dipyridamole, glycoprotein IIb/IIIa platelet membrane receptor antagonist, recombinant hirudin, and thrombin inhibitor. Exemplary antiangioplastic agents include thiphosphoramide. Exemplary antisense agents include oligionucleotides and combinations. Exemplary anticoagulants include hirudin, heparin, synthetic heparin salts and other inhibitors of thrombin. Exemplary antibiotics include vancomycin, dactinomycin (e.g., actinomycin D), daunorubicin, doxorubicin, and idarubicin. Exemplary disintegrins include saxatilin peptide. Derivatives and analogs thereof of these examples are also included.

Other exemplary classes of agents include agents that inhibit restenosis, smooth muscle cell inhibitors, immunosuppressive agents, and anti-antigenic agents.

Exemplary drugs include sirolimus, paclitaxel, tacrolimus, heparin, pimecrolimus, midostaurin, imatinib mesylate (gleevec), and bisphosphonates.

The concentration of the drug in the composition can be tailored depending on the specific target cell, disease extent, lumen type, etc. In one embodiment, the concentration of drug in the lipid film can range from 0.001% to 75% by weight relative to the total weight of the solid film, such as a concentration of 0.1% to 50% by weight relative to the total weight of the solid film. In another embodiment, the concentration of drug in the lipid film can range from 0.01% to 40% by weight, such as a concentration ranging from 0.1% to 20% by weight relative to the total weight of the solid film. In another embodiment, the concentration of drug in the lipid film range from 1% to 50%, 2% to 45%, 5% to 40%, or 10% to 35% by weight, relative to the total weight of the solid film. In another embodiment, the drug load can range from 0.1 ng to 5 μg per mm length of a given stent configuration, such as a drug load ranging from 1 ng to 5 μg, or from 0.1 ng to 1 μg, or from 1 ng to 1 μg, or from 0.1 ng to 100 ng or from 0.1 μg to 5 μg, or from 0.1 μg to 1 μg, or from orfrom 1 μg to 5 μg.

In one embodiment, a biocompatible substrate, such as a ceramic is provided on the medical device to provide a surface that can promote growth of endothelial cells of the vascular intima, i.e., endothelialization. Previously, drug eluting stents have been developed to elute anti-proliferative drugs from a non-degradable aromatic polymer coating and are currently used to further reduce the incidence of restenosis. Commercially available drug eluting stents, such as the Cypher® stent, which elutes sirolimus, and the Taxus® stent, which elutes paclitaxel, do not promote endothelialization, most likely because of the non-degradable polymer.

In one embodiment, upon resorption of the composition (e.g., lipid/drug) by the aqueous solution or body fluid, the surface of the biocompatible ceramic is exposed to the body fluid. Ceramics can persist in the body for one or more years, and a stable, persistent coating is not undesirable in the body since endothelialization has been demonstrated on biocompatible ceramics, such as a hydroxyapatite coating.

In one embodiment, the thickness of the porous substrate coating can be adjusted so that it provides the necessary volume for deposition of the composition comprising one or more lipids and one or more pharmaceutically active agents. The adhesion of the porous substrate coating to the surface of the medical device should be such that the porous substrate does not delaminate from the surface of the medical device during implantation.

In one embodiment, the porous substrate has a thickness of 10 μm or less. In other embodiments, e.g., where the device is an orthopedic implant, the porous substrate can have a thickness ranging from 10 μm to 5 mm, such as a thickness ranging from 100 μm to 1 mm.

In another embodiment, the device is a stent, and the thickness of the substrate is selected to provide a sufficiently flexible coating that stays adhered to the stent even during mounting and expansion of the stent. A typical mounting process involves crimping the mesh-like stent onto a balloon of a catheter, thereby reducing its diameter by 75%, 65%, or even 50% of its original diameter. When the balloon mounted stent is expanded to place the stent adjacent a wall of a body lumen, e.g., an arterial lumen wall, the stent, in the case of stainless steel, can expand to up to twice or even three times its crimped diameter. For example, a stent having an original diameter of 1.7 mm can be crimped to a reduced diameter of 1.0 mm. The stent can then be expanded from the crimped diameter of 1.0 mm to 3.0 mm. Accordingly, in one embodiment, the substrate has a thickness of no more than 2 μm, such as a thickness of no more than 1 μm, or a thickness of no more than 0.5 μm.

In one embodiment, the calcium phosphate in the coating is porous and has a porosity volume ranging from 30 to 70% and an average pore diameter ranging from 0.3 μm to 0.6 μm. In other embodiments, the porosity volume ranges from 30 to 60%, from 40 to 60%, from 30 to 50%, or from 40 to 50%, or even a porosity volume of 50%. In yet another embodiment, the average pore diameter ranges from 0.4 to 0.6 μm, from 0.3 to 0.5 μm, from 0.4 to 0.5 μm, or the average pore diameter can be 0.5 μm. Calcium phosphates displaying various combinations of the disclosed thicknesses, porosity volumes or average pore diameters can also be prepared.

In one embodiment, the substrate is well bonded to the stent surface and neither forms significant cracks nor flakes off the stent during mounting on a balloon catheter and placement in an artery by expansion. In one embodiment, a coating that does not form significant cracks can have still present minor crack formation so long as it measures less than 300 nm, such as cracks less than 200 nm, or even less than 100 nm.

In another embodiment, the coating can withstand a fatigue test to meet the requirements as per the “FDA Draft Guidance for the Submission of Research and Marketing Applications for Interventional Cardiology Devices” that demonstrates the safety of the device from mechanical fatigue failures for at least one year of implantation life. The test is designed to simulate the stent fatigue due to the expansion and contraction of the vessel in which it is implanted. For example, the coated stents can be tested in phosphate buffer saline (PBS) at 37° C.±3 C, with a EnduraTec fatigue testing machine (ElectroForce® 9100 Series, EnduraTec System Corporation, Minnesota, USA) that can simulate the equivalent of one year of in-vivo implantation, e.g., approximately 40 million cycles of fatigue stress, which simulates heart beat rates from 50-100 beats per minute.

In one embodiment, the substrate is a calcium phosphate coating, such as hydroxyapatite. The calcium phosphate coating may be deposited by electrochemical deposition (ECD) or electrophoretic deposition (EPD). In another embodiment the coating may be deposited by a sol gel (SG) or an aero-sol gel (ASG) process. In another embodiment the coating may be deposited by a biomimetic (BM) process. In another embodiment the coating may be deposited by a calcium phosphate cement (CPC) process. In one embodiment of a cement process, a calcium phosphate cement coating with about a 16 nm pore size, a porosity of about 45%, and containing a dispersed or dissolved therapeutic agent, is applied to a stent previously coated with a sub-micron thick coating of sol-gel hydroxyapatite as previously described in U.S. Pat. No. 6,730,324, the disclosure of which is incorporated herein by reference. The resulting coating encapsulates the agent, and agent release is controlled by the dissolution of the coating.

Calcium phosphates, e.g., hydroxyapatite, in the crystalline state can persist on a device for one or more years. Crystalline hydroxyapatite coatings normally release an agent at a rate controlled by pore size and shape, not by dissolution of the coating. However, a stable, persistent calcium phosphate coating, such as a hydroxyapatite coating, is not undesirable in the body since endothelialization has been demonstrated on crystalline hydroxyapatite. In contrast, polymer coatings of prior art drug eluting stents do not promote endothelialization.

Another embodiment provides a metal stent comprising at least one coating covering at least a portion of the stent, the at least one coating having a thickness of no more than 2 μm and comprising:

a porous calcium phosphate having a porosity volume ranging from 30-70% and an average pore diameter ranging from 0.3 μm to 0.6 μm; and at least one pharmaceutically active agent impregnating the porous calcium phosphate,

wherein the coating is free of a polymeric material.

Another embodiment provides a stent comprising:

a porous substrate;

a composition impregnating the porous substrate, the composition comprising at least one pharmaceutically active agent and a polymer-free, bioresorbable carrier.

The porous substrate can be the stent itself or another material covering at least a portion of the stent, e.g., metal oxides, metal carbides, and calcium phosphates.

In one embodiment, a “bioresorbable” as used herein refers to a substance capable of decomposing, degenerating, degrading, depolymerizing, or any other mechanism that allows the carrier to be either soluble in the resulting body fluid or, if insoluble, to be suspended in a body fluid and transported away from the implantation site without clogging the flow of the body fluid. The body fluid can be any fluid in the body of a mammal including, but not limited to, blood, urine, saliva, lymph, plasma, gastric, biliary, or intestinal fluids, seminal fluids, and mucosal fluids or humors. In one embodiment, the biodegradable polymer is soluble, degradable as defined above, or is an aggregate of soluble and/or degradable material(s) with insoluble material(s) such that, with the resorption of the soluble and/or degradable materials, the residual insoluble materials are of sufficiently fine size such that they can be suspended in a body fluid and transported away from the implantation site without clogging the flow of the body fluid. Ultimately, the degraded compounds are eliminated from the body either by excretion in perspiration, urine or feces, or dissolved, degraded, corroded or otherwise metabolized into soluble components that are then excreted from the body.

Exemplary bioresorbable carriers include any polymer-free carriers, such as the lipids disclosed herein and mixtures thereof, or non-lipids, such as pliable materials including azone and hydrocarbons, e.g., mineral oils.

A lipid (such as a triglyceride exemplified by castor oil) may be resorbed at its implantation site by one or more of several mechanisms. It may be solubilized at the molecular level over time in the local body fluid. It may be solubilized one or more molecules at a time into serum albumin, lipoproteins or similar lipid binding proteins in the body fluid. It may be degraded chemically or enzymatically at the implantation site into its more soluble components, e.g., fatty acids and mono- or diglycerides. It may be resorbed as lipid particles or droplets.

In one embodiment, the porosity volume and pore sizes in calcium phosphate coatings can be selected to act as reservoirs for controlling the release of pharmaceutically active agents. In one embodiment, the pharmaceutically active agent is selected from those agents used for the treatment of restenosis, e.g., anti-inflammatory agents, anti-proliferatives, pro-healing agents, gene therapy agents, extracellular matrix modulators, anti-thrombotic agents/anti-platelet agents, antiangioplastic agents, antisense agents, anticoagulants, antibiotics, bone morphogenetic proteins, integrins (peptides), and disintegrins (peptides and proteins), or any agent and mixture thereof disclosed herein. Other exemplary classes of agents include agents that inhibit restenosis, smooth muscle cell inhibitors, immunosuppressive agents, and anti-antigenic agents. Exemplary drugs include sirolimus, paclitaxel, tacrolimus, heparin, pimecrolimus, midostaurin, imatinib mesylate (gleevec), and bisphosphonates.

The release of drugs from prior art polymer coatings for drug eluting stents depend substantially on the rate of diffusion of the drug through the polymer coating. While diffusion may be a suitable mechanism for drug release, the rate of drug release from the polymer coating may be too slow to deliver the desired amount of drug to the body over a desired time. As a result, a significant amount of the drug may remain in the polymer coating. In contrast, one embodiment disclosed herein allows selecting the porosity volume and average pore size to provide pathways for the drug be released from the coating, thereby increasing the rate of drug release compared to a polymer coating. In another embodiment, these porosity properties can be tailored to control the rate of drug release. In one embodiment, at least 50% of the agent is released from the stent over a period of at least 7 days, or at least 10 days and even up to a period of 1 year. In another embodiment, at least 50% of the agent is released from the stent over a period ranging from 7 days to 6 months, from 7 days to 3 months, from 7 days to 2 months, from 7 days to 1 month, from 10 days to 1 year, from 10 days to 6 months, from 10 days to 2 months, or from 10 days to 1 month.

Another embodiment provides a stent comprising:

a porous substrate; and

a composition impregnating at least a portion of the porous substrate, the composition comprising at least one pharmaceutically active agent and a non-particulate bioresorbable carrier.

Another embodiment provides a stent comprising:

a porous substrate covering at least a portion of the stent, the substrate comprising a ceramic selected from metal oxides, metal carbides, and calcium phosphates; and

a composition impregnating at least a portion of the porous substrate, the composition comprising at least one pharmaceutically active agent and a bioresorbable carrier.

In these embodiments, the bioresorbable carrier can include any of the polymer-free carriers disclosed herein, e.g., the lipids disclosed herein and mixtures thereof, or pliable non-lipid materials (e.g., azone, mineral oils), or even bioresorbable polymers. Exemplary bioresorbable polymers include poly(ethylene vinyl acetate), polyanhydrides, polyglycolic acid, collagen, polyorthoesters, polyesters, polyalkylcyanoacrylates, polyorthoesters, polyanhydrides, polycaprolactones, polyurethanes, polyesteramides, polydioxanones, polyacetals, polyketals, polycarbonates, polyorthocarbonates, polyphosphazenes, polyhydroxybutyrates, polyhydroxyvalerates, polyalkylene oxalates, polyalkylene succinates, poly(malic acid), poly(amino acids), polyvinylpyrrolidone, polyvinyl alcohol (PVA), polyalkylene glycols (PAG) such as polyethylene glycol, polyalkylcarbonate, chitin, chitosan, starch, fibrin, polyhydroxyacids such as polylactic acid and polyglycolic acid, poly(lactide-co-glycolide) (PLGA), poly(l-lactide-co-trimethylene carbonate), poly(d,l-lactide-co-trimethylene carbonate), poly(d,l-lactide), poly(d,l-lactide-co-glycolide), polyhydroxycellulose, poly(butyric acid), poly(valeric acid), proteins and polysaccharides such as collagen, hyaluronic acid, albumin, gelatin, cellulose, dextrans, fibrinogen, and blends and copolymers thereof. In one embodiment, the bioresorbable polymer is biocompatible, where a biocompatible polymer is a polymeric material that is compatible with living tissue or a living system, and is sufficiently non-toxic or non-injurious and causes minimal (if any) immunological reaction or rejection.

In one embodiment, a non-particulate carrier has a diameter greater than 500 nm, such as a diameter greater than 1 μm, a diameter greater than 2 μm, a diameter greater than 5 μm, a diameter greater than 10 μm, a diameter greater than 25 μm, a diameter greater than 100 μm, a diameter greater than 500 μm, or even a diameter greater than 1 mm. In another embodiment, a non-particulate carrier has no definable diameter, e.g., a continuous film, or non-continuous film with domains having dimensions greater than 500 nm, e.g., greater than 1 μm, greater than 2 μm, greater than 5 μm, greater than 10 μm, greater than 25 μm, greater than 100 μm, greater than 500 μm, or domains greater than 1 mm.

Another embodiment provides a stent comprising:

a porous substrate covering at least a portion of the stent and comprising a ceramic;

a composition impregnating the porous substrate, the composition comprising at least one pharmaceutically active agent and a polymer-free, bioresorbable carrier.

Another embodiment provides a stent comprising:

a porous metallic substrate;

a composition impregnating the porous substrate, the composition comprising at least one pharmaceutically active agent and a polymer-free, bioresorbable carrier.

In one embodiment, the porous metallic substrate is the stent itself. In another embodiment, the porous metallic substrate covers at least a portion of the stent. In one embodiment, the porous metallic substrate is selected from metals typically used for stents, e.g., stainless steel, CoCr, titanium, titanium alloys, and NiTi.

Another embodiment provides a stent comprising:

a porous polymeric substrate;

a composition impregnating the porous substrate, the composition comprising at least one pharmaceutically active agent and a polymer-free, bioresorbable carrier.

In one embodiment, the stent comprises a porous polymer, and thus offers a porous polymeric surface. In another embodiment, the porous polymeric substrate covers at least a portion of a metallic or polymeric stent. In either embodiment, suitable polymers include any of the non-resorbable and bioresorbable polymers disclosed herein.

Another embodiment provides a stent comprising:

a porous substrate covering at least a portion of the stent and comprising at least one calcium phosphate;

a composition impregnating the porous substrate, the composition comprising at least one pharmaceutically active agent and a bioresorbable carrier, such as a polymer-free bioresorbable carrier.

In one embodiment, the porous substrate comprises hydroxyapatite. In one embodiment, the at least one pharmaceutically active agent is selected from anti-inflammatory agents and anti-proliferative agents. In one embodiment, the at least one pharmaceutically active agent is selected from midostaurin and sirolimus.

Another embodiment provides a stent comprising:

a porous substrate covering at least a portion of the stent and comprising hydroxyapatite;

a composition impregnating the porous substrate, the composition comprising at least one pharmaceutically active agent and a bioresorbable carrier, such as a polymer-free bioresorbable carrier.

In one embodiment, the bioresorbable carrier comprises at least one lipid, such as a triglyceride. In one embodiment, the at least one lipid comprises castor oil.

In one embodiment, the at least one pharmaceutically active agent is selected from anti-inflammatory agents and anti-proliferative agents. In one embodiment, the at least one pharmaceutically active agent is selected from midostaurin and sirolimus.

Another embodiment provides a stent comprising:

a porous substrate covering at least a portion of the stent and having a porosity volume ranging from 30-70% and an average pore diameter ranging from 0.3 μm to 0.6 μm;

a composition impregnating the porous substrate, the composition comprising at least one pharmaceutically active agent and a bioresorbable carrier, such as a polymer-free bioresorbable carrier.

In one embodiment, the porous substrate comprises a ceramic, such as any ceramic disclosed herein, e.g., calcium phosphates. In one embodiment, the porous substrate comprises hydroxyapatite. In one embodiment, the carrier comprises at least one lipid, e.g., a triglyceride. In one embodiment, the at least one lipid comprises castor oil. In one embodiment, the at least one pharmaceutically active agent is selected from anti-inflammatory agents and anti-proliferative agents. In one embodiment, the at least one pharmaceutically active agent is selected from midostaurin and sirolimus.

Another embodiment provides a method of making a coated stent, comprising:

etching a stainless steel stent with a first alkaline solution; electrochemically depositing at least one calcium phosphate to coat at least a portion of the stent to form a coated stent; and

subjecting the coated stent to a second alkaline solution.

In one embodiment, the first alkaline solution is a sodium hydroxide solution. In one embodiment, the sodium hydroxide solution has a sufficient concentration to provide the stainless steel stent surface with roughness features measuring 200 nm or less, such as roughness features measuring 100 nm or less. This roughness improves the adhesion of the calcium phosphate to the stent, as compared to the adhesion to a smooth stent surface. Optionally, after the etching step, the stainless steel stent can be further subjected to heating, such as heating at temperatures ranging from 400° C. to 600° C.

The electrochemical deposition can be varied to achieve the desired porosity features. Variables include current density (e.g., ranging from 0.5-2 mA/cm²), deposition time (e.g., 2 minutes or less, or 1 minute or less), and electrolyte composition, pH, and concentration. Such variables can be manipulated as discussed in Tsui, Manus Pui-Hung, “Calcium Phosphate Coatings on Coronary Stents by Electrochemical Deposition,” M.A.Sc. diss., University of British Columbia, University, 2006, the disclosure of which is incorporated herein by reference.

In one embodiment, the electrochemically deposited calcium phosphate is a mixed-phase coating comprising partially crystalline hydroxyapatite and dicalcium phosphate dihydrate. Substantially pure hydroxyapatite can be achieved by subjecting the coated stent to the second alkaline solution, followed by heating the coated stent at a temperature ranging from 400° C. to 750° C., such as a temperature ranging from 400° C. to 600° C. The phase can be monitored by x-ray diffraction, or other methods known in the art. In one embodiment, the method results in a porous calcium phosphate, such as a porous hydroxyapatite. The porous calcium phosphate (e.g., porous hydroxyapatite) can be stable in body fluid for at least one year, or even for at least two years, thereby allowing sufficient time for endothelialization to occur on the calcium phosphate surface.

In one embodiment a composition ratio of calcium salt and phosphate salt is selected to give a desired calcium phosphate after deposition. For example, a Ca/P ratio can be selected to range from 1.0 to 2.0.

In another embodiment, the release rate of a therapeutic agent by a calcium phosphate coating can be controlled by the bioresorption or biodegradation of the calcium phosphate itself. Bioresorption and biodegradation can be generally controlled by at least one or more of the following factors: (1) physiochemical dissolution, e.g., degradation depending on the local pH and the solubility of the biomaterial; (2) physical disintegration, e.g., degradation due to disintegration into small particles; and, (3) biological factors, e.g., degradation cause by biological responses leading to local pH decrease, such as inflammation.

In one embodiment, the rate of bioresorption or biodegradation is controlled by the solubility properties of the calcium phosphate. In general the more soluble calcium phosphates dissolve more rapidly than the less soluble calcium phosphates. A more soluble, and thus, more rapidly biodegradable, calcium phosphate can slowly be solubilized from the stent, leaving a bare metal stent. Such bare metal stents are known to be compatible with the endothelial cell layer.

The solubility of the calcium phosphate can be dependent on one or more properties such as surface area, density, porosity, composition, Ca/P ratio, crystal structure, and crystallinity. In general amorphous calcium phosphates dissolve faster than partially crystalline calcium phosphates, e.g., mixtures of amorphous and crystalline calcium phosphates, or calcium phosphate displaying poor crystalline structures. Such partially crystalline calcium phosphates generally dissolve faster than all-crystalline calcium phosphates.

In one embodiment, a calcining temperature is selected to give a calcium phosphate. In another embodiment a low calcining temperature is selected to give a partially crystalline calcium phosphate. In another embodiment a low calcining temperature is selected to give a mixture of amorphous and crystalline calcium phosphates. In another embodiment an even lower calcining temperature is selected to give an amorphous calcium phosphate. In another embodiment a low calcining temperature is selected to give a mixture of calcium phosphates.

Amorphous calcium phosphate coatings can be made partially crystalline by heating (calcining) at lower temperatures, e.g., at temperatures ranging from less than 400° C. In one embodiment, the as-deposited calcium phosphate can be too soluble (e.g., dissolving within hours) and can be made more crystalline by heating at higher temperatures, e.g., at temperatures greater than 400° C. Coatings made of the more soluble compounds release a contained agent over a shorter period of time than coatings of the less soluble compounds.

While various variables can have an effect on the biodegradation of calcium phosphate, the general order of solubility at near-neutral pH environment, in one embodiment, is as follows (from highest to lowest):

amorphous calcium phosphate (ACP )>dicalcium phosphate (DCP)>tetracalcium phosphate (TTCP)>octacalcium phosphate (OCP)>alpha-tricalcium phosphate (α-TCP)>beta-tricalcium phosphate (β-TCP)>hydroxyapatite (HAp)

In one embodiment, the coating comprises at least one calcium phosphate selected from octacalcium phosphate, α- and β-tricalcium phosphates, amorphous calcium phosphate, dicalcium phosphate, calcium deficient hydroxyapatite, and tetracalcium phosphate, e.g., the coating can comprise a pure phase of any of the calcium phosphates or mixtures thereof, or even mixtures of these calcium phosphates with hydroxyapatite.

In another embodiment, the solubility of the calcium phosphate can be selected based on their inherent solubility, or Kᵢₚ, as reported by Dorozhkin and Epple (Biological and medical significance of calcium phosphates, Angew. Chem. Int. Ed. Eng. 41: 3130-3146 (2002)). Kᵢₚ is the negative logarithm of the ion product with concentrations in M. Kᵢₚ values for various calcium phosphates are listed in Table 1 below.

TABLE 1
Solubility of calcium phosphates in water at 25° C.

| | | Solubility |
|---|---|---|
| Ca/P | | (25° C., |
| ratio | Compound | log(Kᵢₚ)) |
| 0.5 | Monocalcium phosphate monohydrate, | 1.14 |
| | Ca(H₂PO₄)₂•H₂O | |
| 0.5 | Monocalcium phosphate anhydrate, Ca(H₂PO₄)₂ | 1.14 |
| 1.0 | Dicalcium phosphate, Ca(HPO₄)•H₂O | 6.59 |
| 1.0- | Dicalcium phosphate anhydrate, Ca(HPO₄) | 6.90 |
| 1.23 | Octacalcium phosphate, Ca₃(HPO₄)(PO₄)₂ | 96.6 |
| 1.33 | α-calcium phosphate, α-Ca₃(PO₄)₂ | 25.5 |
| 1.5 | β-tricalcium phosphate, β-Ca₃(PO₄)₂ | 28.9 |
| 1.2-2.2 | Amorphous calcium phosphate, Ca₃(PO₄)₂•nH₂O) | ~30 |
| 1.5-1.67 | Calcium deficient hydroxyapatite, | ~85.1 |
| | Ca₁₀₋ₓ(HPO₄)ₓ(PO₄)₆₋ₓOH)₂₋ₓ(x < 1) | |
| 1.67 | Hydroxyapatite, Ca₁₀(PO₄)₆(OH)₂ | 118.8 |
| 2.0 | Tetracalcium phosphate, Ca(PO₄)₂O | 38-44 |

Accordingly, one embodiment provides a metal stent comprising at least one coating covering at least a portion of the stent, the at least one coating comprising:

at least one calcium phosphate deposited on the metal stent, the at least one calcium phosphate having sufficient solubility in water such that the coating has a water solubility, as determined by −log(Kᵢₚ), of less than 100.

Another embodiment provides a metal stent comprising at least one coating covering at least a portion of the stent, the at least one coating comprising:

at least one porous calcium phosphate deposited on the metal stent, the at least one porous calcium phosphate having sufficient solubility in water such that the coating has a water solubility, as determined by −log(Kᵢₚ), of less than 100; and

at least one pharmaceutically active agent impregnating the at least one porous calcium phosphate.

In one embodiment, the at least one pharmaceutically active agent is combined with a carrier, such as any bioresorbable carrier disclosed herein.

In any of these embodiments, calcium phosphates can be made more soluble (faster resorption, faster drug release) by partial replacement of calcium with other ions such as sodium, potassium, and/or magnesium, and/or by partial replacement of phosphate with carbonate, or chloride.

In one embodiment, a mixture of dicalcium phosphate dihydrate and poorly crystalline hydroxyapatite can be electrochemically deposited on a stent. This coating can dissolve at neutral pH in 40 minutes. In another embodiment, conversion of this coating to hydroxyapatite by treatment with alkali gives a coating which dissolves in 6.5 hours. In another embodiment heating the alkali treated coating to 500° C. gives a crystalline hydroxyapatite coating which dissolves in >4 weeks.

In one embodiment, dissolution tests can be performed with Varian dissolution apparatus (Varian VK750D, Varian Inc., California, USA). Variables include precise bath temperature and rotation speed control, and the use of seal bottles to prevent dissolution media from evaporation. Dissolution tests can be conducted at a bath temperature of 37° C. and rotation speed at 20 rpm. Phosphate buffer saline (PBS), which is isotonic, can be used as the dissolution media to maintain constant pH (7.4). The PBS solution can contain 10 mM phosphate, 140mM NaCl, and 3mM KCl. For example, ECD coated stents can be placed into dissolution apparatus with sealed bottles of 10 mL PBS, and ECD coated stents were weighted over a period of 30 minutes to 4 weeks to determine the weight loss of the coating due to dissolution.

In one embodiment at least one calcium phosphate is deposited on a stent as a single layer. In another embodiment a single calcium phosphate is deposited as multiple layers. In another embodiment a calcium phosphate is deposited in one layer and one or more layers of one or more other calcium phosphates can be successively deposited over the first layer.

Another embodiment provides a method of treating at least one disease or condition associated with restenosis, using either a stent coated with at least one porous calcium phosphate that is stable to resorption, allowing the drug to be released through the pores of the calcium phosphate. In another embodiment, the stent is coated with a porous calcium phosphate that is resorbed relatively quickly to release the drug that impregnates the calcium phosphate.

After or during drug release, another embodiment exposes a surface that promotes endothelialization. In one embodiment the method comprises the steps of:

implanting in a subject in need thereof a metal stent comprising at least one coating covering at least a portion of the device, the at least one coating comprising:
at least one porous calcium phosphate having a porosity volume ranging from 30-60% and an average pore diameter ranging from 0.3 μm to 0.6 μm, and
at least one pharmaceutically active agent impregnating the at least one porous calcium phosphate;

releasing from the coating the least one pharmaceutically active agent by allowing the at least one porous calcium phosphate to dissolve; and

completely dissolving the at least one porous calcium phosphate to expose a metal surface of the metal stent.

In this embodiment, endothelialization occurs on the exposed metal surface of the metal stent, which is also known to be non-thrombogenic. Thus, the step of completely dissolving occurs within a period of less than 6 months, such as a period of less than 2 months, a period of less than one month, or a period of less than 2 weeks.

Another embodiment provides a method of treating at least one disease or condition associated with restenosis, comprising:

implanting in a subject in need thereof a metal stent comprising at least one coating covering at least a portion of the device, the at least one coating comprising:
at least one porous calcium phosphate having a porosity volume ranging from 30-60% and an average pore diameter ranging from 0.3 μm to 0.6 μm, and
at least one pharmaceutically active agent impregnating the at least one porous calcium phosphate;

releasing from the coating the least one pharmaceutically active agent by allowing the at least one porous calcium phosphate to dissolve; and

allowing the at least one porous calcium phosphate to remain on the stent for a period of at least six months.

In this embodiment, endothelialization occurs on the surface of the calcium phosphate. In one embodiment, the calcium phosphate remains on the stent for a period of at least one year, at least two years, or even at least three years.

### EXAMPLES

The Examples disclosed herein describe the use of hydroxyapatite-coated stents as prepared in U.S. Provisional Application No. 60/978,988, filed Oct. 10, 2007, the disclosure of which is incorporated herein by reference. It would be understood by one of ordinary skill in the art that the Examples below can also be performed with the calcium phosphate or hydroxyapatite-coated stents, such as those devices described in U.S. Patent Publication No. 2006/0134160, the disclosure of which is incorporated herein by reference.

### Example 1

This Example describes a stent pretreatment process and deposition of hydroxyapatite on the stent, as disclosed in Tsui, Manus Pui-Hung, “Calcium Phosphate Coatings on Coronary Stents by Electrochemical Deposition,” M.A.Sc. diss., University of British Columbia, University, 2006, the disclosure of which is incorporated herein by reference.

The stent used was a 316L stainless steel stent measuring 14 mm in length and a 0.85 mm outer radius. The stent surface was electro-polished, then cleaned in ultrasonic bath, with distilled water and then with ethyl alcohol. The stent was then soaked in 10N NaOH (aq) at 75° C. for 15 hours and subsequently heat-treated at 500° C. for 20 minutes. The heat treatment is optional and the micro-etched stent may be also coated without it.

Electrochemical deposition of calcium phosphate was performed with 400 mL of electrolyte consisting of 0.02329M Ca(NO₃)₂.4H₂O and 0.04347M NH₄H₂PO₄ at 50° C. The pretreated stent was used as the cathode and a nickel ring was used as the anode. When a 0.90 mA current was applied for 60 seconds, a thin film of hydroxyapatite coating was deposited on the stent. In other embodiments, a current density of 0.5-2 mA/cm² can be used depending on the stent size. The coated stent was then washed with running distilled water for 1 minute and air dried for 5 minutes.

The stent was then subjected to a post-treatment process of soaking the stent in 0.1N NaOH (aqueous) solution at 75° C. for 24 hours, followed by an ultrasonical cleaning with distilled water and a heat treatment at 500° C. for 20 minutes.

The coating uniformly covered the stent and the thickness is ˜0.5 um. The surface morphology of the coating remained unchanged, as compared to the electrochemically deposited hydroxyapatite coating on an un-oxidized stent. An expansion test was performed after the electrochemically deposited hydroxyapatite coated pre-oxidized stent had been air dried. An Encore™ 26 INFLATION DEVICE KIT was used to inflate the catheter to 170 psi. The expanded stent was observed under SEM. No separation of the coating was visible even in the areas of the highest strain due to the expansion, for magnifications up to 10,000×. The stent strain was accommodated by the coating through nano-size localized cracking, not visible under the microscope.

### Example 2

This Example describes the preparation of HAp coated stents containing sirolimus in a castor oil vehicle.

Castor oil (1000 mg) was added to 9000 mg of ethanol and mixed to give a clear solution. Sirolimus (100 mg) was added to 660 mg of the above solution and mixed. 2.0 g of ethanol was added to the sirolimus mixture and stirred to give a clear solution. An HAp coated stent (14 mm in length, with a 0.85 mm outer radius) prepared according to Example 1 was weighed and then dipped into the clear sirolimus solution in a vacuum chamber. The chamber was evacuated until a pressure of 20 mm Hg was reached. The vacuum was released and the stent was placed onto a mandrel and spun at 5000 rpm for 10 seconds. The stent was then dried under a vacuum of 30 mm Hg for 12 hours at ambient temperature and weighed. The amount of sirolimus in the coating was calculated to be 30 μg.

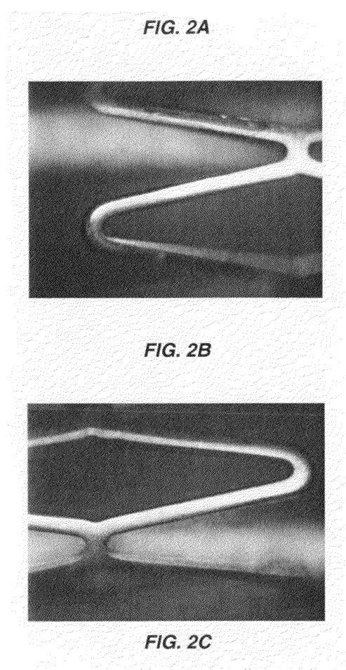

FIGS. 2A-2C are photographs of the coated stent showing the stent morphology. The consistency of the coating is apparent with no observable flaking or cracking.

### Example 3

This Example describes the monitoring of drug release over time for the coated stent of Example 2.

Coated stents prepared according to Example 2 were placed in 0.02% sodium dodecyl sulfate (SLS) in PBS (9 mL), which in turn were placed in a 22° C. rotating water bath. At various time intervals the liquid is replaced with the used liquid being taken for further analysis using an HPLC method. The cumulative amount of drug released is calculated as follows:

% Cumulative drug release=(sum of all drug released prior to and at the current interval)/(total drug in coating by wt.)

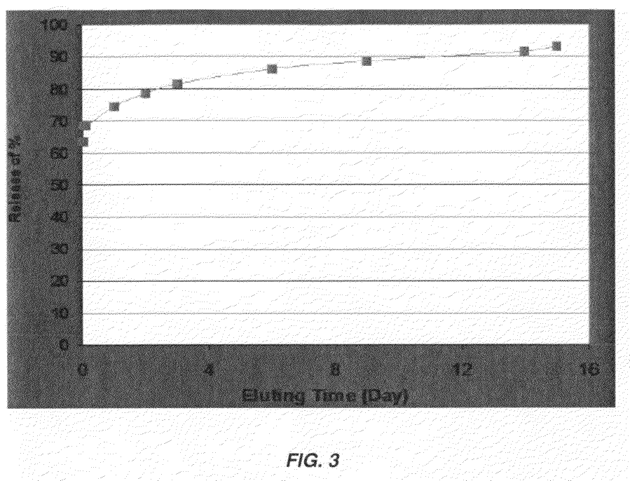

As a comparison, a porous hydroxyapatite coated stent 1 was further coated with sirolimus only, i.e., without a lipid carrier. FIG. 3 is a plot of cumulative % sirolimus release (y-axis) versus time of elution (x-axis). FIG. 3 shows an initial burst release of 70% the total amount of sirolimus. Moreover, approximately 80% of the drug is released within a few days. This dosage course is not suitable for treating the late stent thrombosis that often accompanies stent implantation.

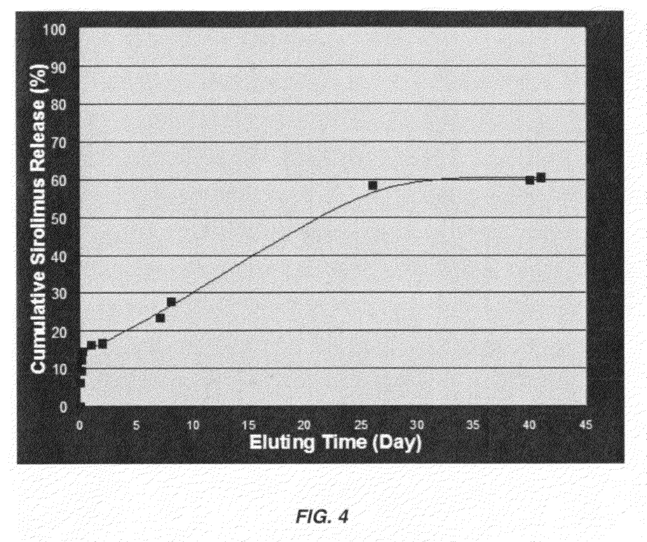

In contrast, the analogous plot (FIG. 4, cumulative % drug release (y-axis) versus time of elution (x-axis)) for the coated stent of the present Example shows a substantially reduced burst release, in which only 10-15% of the drug was released immediately. Moreover, only 20% of the drug was released within 5 days, and 60% of the drug was released within 25 days. This plot indicates that the hydroxyapatite-coated stent impregnated with sirolimus and castor oil is suitable for sustained drug delivery and treatment of late stent thrombosis.

### Example 4

This Example describes the procedure for determining late lumen loss and acute lumen gain in normal coronary arteries of pigs implanted with HAp coated stent of Example 2 containing castor oil and sirolimus compared to the Cypher™ stent containing sirolimus.

Animal preparation. Experiments were performed in juvenile Yorkshire-Landrace swine (25-30 kg). Starting one day before the procedure, 300 mg clopidogrel and 300 mg acetylsalicylic acid were administered orally. After an overnight fast the animals were sedated with 20 mg/kg ketamine hydrochloride and midazolam. After induction of anaesthesia with thiopental (12 mg/kg) and following endotracheal intubation, the pigs were connected to a ventilator which administered a mixture of oxygen and nitrous oxide (1:2 v/v). Anaesthesia was maintained with 0.5-2.5 vol % isoflurane. Antibiotic prophylaxis was administered by an intramuscular injection. Under sterile conditions an arteriotomy of the left carotid artery was performed and a 8F introduction sheath was placed. Acetyl salicylic acid (250 mg) and 10.000 IU heparin sodium was administered. After intraarterial administration of 2 mg isosorbide dinitrate, coronary angiography was performed in two orthogonal views using a non-ionic contrast agent (iodixanol).

Vascular Interventions. From the angiograms, analyzed on-line using a quantitative angiography analysis system, arterial segments of 2.5-3.2 mm in diameter were selected in each of the coronary arteries. Stents were placed with a balloon-artery ratio of 1.1 in a random block design as described before. After repeat angiography of the stented arteries, the guiding catheter and the introducer sheath were removed, the arteriotomy repaired and the skin closed in two layers. The animals were allowed to recover from anaesthesia, while post procedure acetyl salicylic acid, 300 mg, and clopidogrel, 75 mg, were administered daily.

Group size: Group size was calculated using the data of the earlier coronary implants of the stents at the Thoraxcenter. For a 40% difference in neointimal thickness compared to controls, a “paired T-test for sample size” (Sigmastat, Jandel Scientific Software) with a power of 0.8 results in a sample size of 13 coronary implants per group.

Follow-up: At 28 days follow-up, angiography of the stented arteries were performed using the same settings of the X-ray equipment as during implantation, to assess luminal narrowing within the treated segments. Thereafter the coronary arteries were in situ pressure fixed for histology.

Experimental Groups and group size.
ECD-HAP coated stent+30 μg sirolimus in castor oil vehicle: n=13 coronary implants
Cypher™ stent (140 μg of sirolimus): n=13 coronary implants

Number of animals. Thirteen (13) pigs were used in the study.

Routine Histology. All tissue samples were processed for light microscopy to check for any abnormal vascular reaction to the interventions and for a general assessment of the histological appearance. Sections were stained with haematoxylin-eosin as a routine stain and resorcin-fuchsin as an elastin stain. Specific stains were performed as needed.

Quantitative Histology. Inflammatory and degenerative changes were assessed semi-quantitatively as none (0), mild (1), moderate (2) or severe (3).

Immunocytochemistry. Healing and organization of the stented segments will also be assessed by specific stains for white blood cells (CD45), fibrinoid (glycophorin), smooth muscle cells (actin), and endothelial cells (e.g. lectin). When appropriate parameters will be quantified.

Morphometry. Morphometric analysis to determine intimal and medial thickness and area were performed on elastin stained sections by tracing the external and internal elastic laminae and the endothelial lining using an image analysis system. The media is defined as the layer between the internal and external elastic laminae. The distance between the endothelial lining and the internal elastic lamina was taken as the thickness of the intima.

Endpoints
Morphometry: Neo-intimal area, medial area, adventitial area, neointimal thickness, medial thickness, adventitial thickness.
Histology: Injury score, inflammatory score, vascular healing, endothelialization
Angiography: Mean luminal diameter (stented segment), late loss.

### Example 5

This Example describes the analysis of the experiments and measurements described in Example 4.

Angiography. The angiography results of Example 4 are given in Table 2 below.

Pre=artery diameter (mm) at baseline angiography; Max Stent=maximum stent expansion diameter (mm) during placement; B/A ratio=balloon artery ratio during prior injury; S/A ratio=stent artery ratio; Post=artery diameter (mm) after stent implantation; FU=artery diameter (mm) after follow-up; LL=late lumen loss (mm, FU-Post); AG=acute lumen gain (mm, Post-Pre).

TABLE 2
Angiographic results from the HAp-ECD-sirolimus and
Cypher stents of Example 4.

| | | | | | | | |
|---|---|---|---|---|---|---|---|
| | Max | Stent-Artery | | | % | | |
| Pre | Stent | Ratio | Post | FU | Recoil | LL | AG |

STENT HAP

| | | | | | | | | |
|---|---|---|---|---|---|---|---|---|
| mean | 2.69 | 2.93 | 1.08 | 2.80 | 2.57 | 4.20 | 0.23 | 0.11 |
| stdev | 0.27 | 0.28 | 0.05 | 0.25 | 0.19 | 5.57 | 0.21 | 0.09 |
| count | 13 | | | | | | | |

Stent Cypher

| | | | | | | | | |
|---|---|---|---|---|---|---|---|---|
| mean | 2.67 | 2.90 | 1.09 | 2.76 | 2.45 | 4.50 | 0.32 | 0.09 |
| stdev | 0.20 | 0.26 | 0.05 | 0.21 | 0.27 | 2.84 | 0.23 | 0.08 |
| count | 12 | | | | | | | |

Morphometry of the experiment of Example 4. Table 3 below gives the histomorphometry results from the HAp-ECD-sirolimus and Cypher stents of Example 4. Neointima thickness and area, media thickness, and lumen area were not significantly different between the HAP-ECD stent with 30 ug sirolimus and Cypher with 140 ug sirolimus.

TABLE 3
Histomorphometry results from the HAp-ECD-sirolimus and Cypher
stents of Example 4.

| | HAp-ECD | |
|---|---|---|
| | Sirolimus | Cypher ™ |
| Injury score | 0.27 +/− 0.53 | 0.38 +/− 0.49 |
| NI thickness (μm) | 0.23 +/− 0.09 | 0.28 +/− 0.1 |
| Media (μm) | 0.057 +/− 016 | 0.060 +/− 0.04 |
| Lumen area (mm2) | 6.8 +/− 1.3 | 5.8 +/− 0.8 |
| NI area (mm2) | 1.34 +/− 0.83 | 1.41 +/− 0.57 |

NI - neointima thickness over stent strut

Both coatings performed similarly. Statistical analysis showed no difference in quantitative tissue response between the HAp-ECD-sirolimus and the Cypher™ stent.

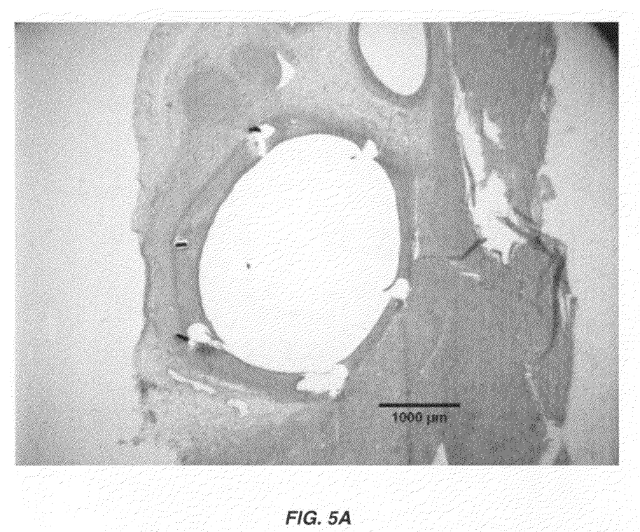

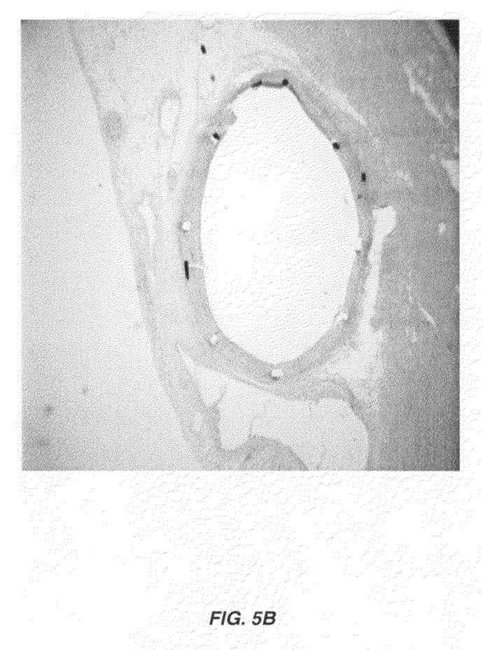

Qualitative histological analysis of the experiment of Example 4. There were two groups: HAp-ECD-sirolimus and Cypher™. FIGS. 5A and 5B show the typical histology of the implanted Cypher™ and the ECD-HAP sirolimus stent. The median sections of lower anterior descending (LAD) arteries are shown for Cypher™ (FIG. 5A) and from the ECD-HAP sirolimus stent (FIG. 5B). Specifically, FIG. 5B shows the histology of an implanted stent coated with hydroxyapatite and sirolimus, as described in Example 3, both after 28 days of implantation in the lower anterior descending artery of a pig. In these single micrographs, the HAp-sirolimus stent presents a thin neointima without major inflammation. The HAp-ECD-sirolimus coated stent showed that, in general, the border zone between intima and media contained areas that were relatively acellular. These areas also contained variable amounts of fibrinoid material and closely packed erythrocytes. The luminal aspect of the intima showed a more normal neointima with partly raised endothelium and adherent leucocytes. There was some inflammation, with a few eosinophils.

Cypher™. This group showed a minimal to moderate neointimal thickening with a reasonable layer of endothelium. In a few cases unhealed struts were observed with a granular neointima, eosinophils and scant endothelium. Again the intima-media border zone contained areas of fibrinoid and erythrocytes and was partially acellular with granular or amorphous material. In areas of abundant neointima and extracellular matrix, vacuoles indicative of cell death were found. In case of inflammation (complete or partial) eosinophils were always present, also luminally.

Based on the histology and the angiography, the stent of Example 4 was equally effective as the Cypher stent at a much lower dose (e.g., 30 μg versus 140 μg for Cypher).

### Example 6

This Example describes human clinical trials performed with the HAp coated stent of Example 2. In this Example, stents of 19 mm in length and 3.0 and 3.5 mm in diameter stents were loaded with 55 and 58 μg sirolimus, respectively.

Stents were implanted into sixteen patients with a single de novo lesion in a coronary artery, fifteen with a single stent each and one with four stents, two of which were study stents and two of which were regular bare metal stents. Lesions were evaluated by quantitative coronary angiography (QCA) and intravascular ultrasound (IVUS). The primary efficacy endpoint was in-stent lumen loss, as assessed by QCA. Before implantation, the average minimum lumen diameter (MLD) in the lesion was 0.99±0.30 mm and the average % diameter stenosis was 62.8±10.3%.

All patients were evaluated immediately after the implantation procedure and then at an interim time point of 4 months by quantitative coronary angiography (QCA) and intravascular ultrasound (IVUS). Evaluation will be repeated at 9 months. Implantation of the stents of increased the preprocedural minimum lumen diameter from 0.99±0.30 mm to 2.62±0.33 mm and reduced the % diameter stenosis from 62.8±10.3% to 3.3±8.1% within the in-stent vessel length. At 4 months follow-up of 13 patients the in-stent minimum lumen diameter was 2.34±0.36 mm and the % diameter stenosis was 10.4±8.1%. The late in-stent lumen loss was 0.27±0.27 mm. These and the results of other measurements are shown in Table 4.

TABLE 4
Clinical results of quantitative coronary angiography of the implantation
of 13 lipid-sirolimus-hydroxyapatite coated stents of Example 3 in 13 patients.

| | | Postprocedure | | 4 Month Follow up | |
|---|---|---|---|---|---|
| Variable (N = 13) | Preprocedure | In-Stent | In-Lesion | In-Stent | In-Lesion |
| Lesion Length | 9.82 ± 1.97 | | | | |
| Reference | 2.77 ± 0.30 | | | | |
| diameter | | | | | |
| MLD, mm | 0.99 ± 0.30 | 2.62 ± 0.33 | 2.20 ± 0.33 | 2.34 ± 0.36 | 2.02 ± 0.37 |
| % Diameter | 62.8 ± 10.3 | 3.3 ± 8.1 | 18.9 ± 8.7 | 10.4 ± 8.1 | 23.2 ± 8.7 |
| stenosis | | | | | |
| Late lumen | NA | NA | NA | 0.27 ± 0.27 | 0.18 ± 0.31 |
| loss, mm | | | | | |
| Acute gain, | NA | 1.63 ± 0.36 | 1.21 ± 0.39 | | |
| mm | | | | | |
| Restenosis, % | NA | NA | NA | 0.0 | 0.0 |

The IVUS volumetric measurements in Table 5 showed minimal or insignificant changes in vessel volume, stent volume and lumen volume from the postprocedure to the 4 month follow-up. Percentage stent obstruction was 2.8%±2.4.

TABLE 5
IVUS parameters at baseline (postprocedure) and 4 month follow-up.

| | | 4 Month Follow-Up, |
|---|---|---|
| IVUS Variables | Baseline, N = 13 | N = 13 |
| Vessel volume (mm³) | 276.7 ± 117.1 | 276.6 ± 84.8 |
| Stent volume (mm³) | 145.7 ± 14 | 142 ± 0.5 |
| Lumen volume (mm³) | 145.8 ± 47.5 | 138.8 ± 33.5 |
| NIH volume (mm³) | N/A | 4.1 ± 3.4 |
| Mallapposition volume (mm³) | 0.15 ± 0.5 | 0.09 ± 0.3 |
| % Stent obstruction | N/A | 2.8 ± 2.4 |

These results show that the lipid-sirolimus-hydroxyapatite coated stents are comparable to current drug-eluting stents. Additionally, the bioabsorbable, polymer-free hydroxyapatite coating may allow endothelialization on the stent and may prevent the late, in-stent thrombosis associated with current drug-eluting stents. The average in-lesion late lumen loss can range from 0.00 to 0.50 mm.

1. A stent comprising:
a porous substrate; and
at least one composition impregnating at least a portion of the porous substrate, wherein the composition comprises at least one pharmaceutically effective agent and at least one lipid.

2. The stent of claim 1, wherein the porous substrate comprises a material that covers at least a portion of the stent.

3. The stent of claim 2, wherein the material comprises a ceramic.

4. The stent of claim 3, wherein the ceramic is selected from calcium phosphates and metal oxides.

5. The stent of claim 3, wherein the ceramic is selected from calcium phosphates.

6. The stent of claim 5, wherein the calcium phosphates comprise hydroxyapatite.

7. The stent of claim 1, wherein the at least one lipid is selected from monoglycerides, diglycerides, triglycerides, ceramides, sterols, sterol esters, waxes, tocopherols, monoalkyl-diacylglycerols, fatty alcohols comprising a hydrocarbon chain of at least 8 carbon atoms, N-monoacylsphingosines, N,O-diacylsphingosines, and triacylsphingosines.

8. The stent of claim 7, wherein the fatty alcohols are selected from C₈-C₃₀ fatty alcohols.

9. The stent of claim 7, wherein the fatty alcohols are selected from C₁₂-C₃₀ fatty alcohols.

10. The stent of claim 7, wherein the monoglycerides, diglycerides, and triglycerides are derived from fatty acids having a chain length of at least 4 carbon atoms.

11. The stent of claim 7, wherein the monoglycerides, diglycerides, and triglycerides are derived from fatty acids having a chain length of at least 8 carbon atoms.

12. The stent of claim 7, wherein the monoglycerides, diglycerides, and triglycerides are derived from fatty acids having a chain length of at least 12 carbon atoms.

13. The stent of claim 1, wherein the at least one lipid is selected from vegetable oils, animal oils, and synthetic lipids.

14. The stent of claim 1, wherein the at least one lipid is selected from triglycerides and vegetable oils.

15. The stent of claim 1, wherein the at least one lipid is selected from phospholipids, fatty acids and fatty amines.

16. The stent of claim 15, wherein the phospholipids are selected from diacylglycerophosphates, monoacylglycerophosphates, cardiolipins, plasmalogens, sphingolipids and glycolipids.

17. The stent of claim 15, wherein the fatty acids and fatty amines have a chain length of at least 8 carbon atoms.

18. The stent of claim 15, wherein the fatty acids and fatty amines have a chain length of at least 12 carbon atoms.

19. The stent of claim 1, wherein no more than 10% by weight of the at least one lipid is soluble in water.

20. The stent of claim 1, wherein no more than 5% by weight of the at least one lipid is soluble in water.

21. The stent of claim 1, wherein, no more than 3% by weight of the at least one lipid is soluble in water.

22. The stent of claim 1, wherein the at least one lipid is selected from soybean oil, cottonseed oil, rapeseed oil, sesame oil, corn oil, peanut oil, safflower oil, fish oil, triolein, trilinolein, tripalmitin, tristearin, trimyristin, triarachidonin, castor oil, cholesterol, and cholesterol derivatives such as cholesteryl oleate, cholesteryl linoleate, cholesteryl myristate, cholesteryl palmitate, cholesteryl arachidate.

23. The stent of claim 1, wherein the at least one lipid is selected from fatty acids, fatty amines, and neutral lipids.

24. The stent of claim 1, wherein the at least one pharmaceutically active agent is chosen from anti-inflammatory agents, anti-proliferatives, pro-healing agents, gene therapy agents, extracellular matrix modulators, anti-thrombotic agents, anti-platelet agents, antisense agents, anticoagulants, antibiotics.

25. The stent of claim 1, wherein the at least one pharmaceutically active agent is selected from anti-proliferative agents and anti-inflammatory agents.

26. The stent of claim 5, wherein the at least one pharmaceutically active agent is selected from anti-proliferative agents and anti-inflammatory agents.

27. The stent of claim 6, wherein the at least one pharmaceutically active agent is selected from anti-proliferative agents and anti-inflammatory agents.

28. The stent of claim 1, wherein the at least one pharmaceutically active agent is selected from paclitaxel, sirolimus, everolimus, tacrolimus, biolimus, pimecrolimus, midostaurin, bisphosphonates, heparin, gentamycin, and matinib mesylate.

29. The stent of claim 5, wherein the at least one pharmaceutically active agent is selected from paclitaxel, sirolimus, everolimus, tacrolimus, biolimus, pimecrolimus, midostaurin, bisphosphonates, heparin, gentamycin, and matinib mesylate.

30. The stent of claim 6, wherein the at least one pharmaceutically active agent is selected from paclitaxel, sirolimus, everolimus, tacrolimus, biolimus, pimecrolimus, midostaurin, bisphosphonates, heparin, gentamycin, and matinib mesylate.

31. The stent of claim 29, wherein the at least one lipid is selected from soybean oil, cottonseed oil, rapeseed oil, sesame oil, corn oil, peanut oil, safflower oil, fish oil, triolein, trilinolein, tripalmitin, tristearin, trimyristin, triarachidonin, castor oil, cholesterol, and cholesterol derivatives such as cholesteryl oleate, cholesteryl linoleate, cholesteryl myristate, cholesteryl palmitate, cholesteryl arachidate.

32. The stent of claim 30, wherein the at least one lipid is selected from soybean oil, cottonseed oil, rapeseed oil, sesame oil, corn oil, peanut oil, safflower oil, fish oil, triolein, trilinolein, tripalmitin, tristearin, trimyristin, triarachidonin, castor oil, cholesterol, and cholesterol derivatives such as cholesteryl oleate, cholesteryl linoleate, cholesteryl myristate, cholesteryl palmitate, cholesteryl arachidate.

33. The stent of claim 1, wherein the composition is released from the stent in the form of films, liposomes, nanocapsules, microcapsules, microdroplets, nanodroplets, microspheres, nanospheres, micelles, and combinations thereof.

34. A medical device, comprising at least one coating covering at least a portion of the device, the at least one coating comprising:
a porous substrate;
a composition impregnating at least a portion of the porous substrate, the composition comprising at least one pharmaceutically effective agent and at least one lipid selected from fatty acids, fatty amines, and neutral lipids.

35. The device of claim 34, wherein the neutral lipid is selected from monoglycerides, diglycerides, triglycerides, ceramides, sterols, sterol esters, waxes, tocopherols, monoalkyl-diacylglycerols, fatty alcohols comprising a hydrocarbon chain of at least 8 carbon atoms, N-monoacylsphingosines, N,O-diacylsphingosines, and triacylsphingosines.

36. The device of claim 35, wherein the neutral lipid is selected from monoglycerides, diglycerides, triglycerides.

37. The device of claim 35, further comprising at least one additional lipid selected from phospholipids, glycolipids, sphingomyelins, cerebrosides, gangliosides, and sulfatides.

38. The device of claim 34, wherein the at least one coating is free of a polymer.

39. The device of claim 34, wherein the porous substrate is chosen from at least one ceramic.

40. The device of claim 39, wherein the at least one ceramic is selected from metal oxides and calcium phosphates.

41. The device of claim 40, wherein the at least one ceramic is selected from calcium phosphates.

42. The device of claim 41, wherein the calcium phosphates comprise hydroxyapatite.

43. The device of claim 34, wherein the ceramic has a thickness of no more than 1 μm.

44. The device of claim 34, wherein the at least one pharmaceutically active agent is chosen from anti-inflammatory agents, anti-proliferatives, pro-healing agents, gene therapy agents, extracellular matrix modulators, anti-thrombotic agents, anti-platelet agents, antisense agents, anticoagulants, antibiotics.

45. The device of claim 44, wherein the at least one pharmaceutically effective agent is selected from anti-proliferative agents and anti-inflammatory agents.

46. The device of claim 34, wherein the at least one pharmaceutically active agent inhibits restenosis.

47. The device of claim 34, wherein the at least one pharmaceutically active agent is selected from smooth muscle cell inhibitors, and immunosuppressive agents.

48. The device of claim 34, wherein the at least one pharmaceutically active agent is selected from sirolimus, paclitaxel, tacrolimus, heparin, pimecrolimus, imatinib mesylate, gentamycin, and midostaurin.

49. The device of claim 34, wherein the ceramic is bioresorbable and releases the at least one pharmaceutically active agent contacting the ceramic upon resorption of the ceramic.

50. The device of claim 34, wherein the device is an implantable medical device.

51. The device of claim 34, wherein the device is a stent.

52. A method of treating at least one disease or condition comprising:
implanting in a subject in need thereof a stent comprising:
a porous substrate;
a composition coating or impregnating at least a portion of the porous substrate, the composition comprising at least one pharmaceutically effective agent and at least one lipid; and
releasing from the device the at least one pharmaceutically active agent.

53. The method of claim 52, wherein the at least one pharmaceutically active agent is released from the stent encapsulated in liposomes, nanocapsules, microcapsules, microdroplets, nanodroplets, microspheres, nanospheres, micelles, and combinations thereof.

54. The method of claim 52, wherein the at least one pharmaceutically active agent is released from the device associated with particles comprising the at least one lipid.

55. The method of claim 54, wherein the particles are selected from liposomes, nanocapsules, microcapsules, microdroplets, nanodroplets, microspheres, nanospheres, and micelles.

56. The method of claim 54, wherein the at least one pharmaceutically active agent is released from the device encapsulated in the particles.

57. The method of claim 54, wherein the particles have a size distribution such that at least 5% of the particles are greater than 1 μm.

58. The method of claim 54, wherein the particles greater than 1 μm are capable of being taken up by macrophages.

59. The method of claim 52, wherein the at least one pharmaceutically active agent is selected from anti-proliferative agents and anti-inflammatory agents.

60. A method of treating at least one disease or condition comprising:
implanting in a subject in need thereof a medical device comprising:
a porous substrate;
a composition impregnating at least a portion of the porous substrate, the composition comprising at least one pharmaceutically effective agent and at least one lipid selected from fatty acids, fatty amines, and neutral lipids; and
releasing from the device the at least one pharmaceutically active agent.

61. The method of claim 60, wherein the at least one pharmaceutically active agent is selected from anti-proliferative agents and anti-inflammatory agents.

62. The method of claim 60, wherein the at least one disease or condition is associated with restenosis.

63. A stent comprising:
a porous substrate;
a composition impregnating at least a portion of the porous substrate, the composition comprising at least one pharmaceutically active agent and a polymer-free, bioresorbable carrier.

64. A stent comprising:
a porous substrate covering at least a portion of the stent, the substrate comprising a ceramic selected from metal oxides, metal carbides, and calcium phosphates; and
a composition impregnating at least a portion of the porous substrate, the composition comprising at least one pharmaceutically active agent and a bioresorbable carrier.

65. The stent of claim 64, wherein the bioresorbable carrier is selected from polymers and lipids.
